(19) United States
(12) Patent Application Publication
(10) Pub. No.: US 2020/0146841 A1
(43) Pub. Date: May 14, 2020

(54) DEVICES, SYSTEMS, AND METHODS FOR STABILIZATION OF INTERVERTEBRAL CAGES

(71) Applicant: Juan F. Ronderos, Mobile, AL (US)

(72) Inventor: Juan F. Ronderos, Mobile, AL (US)

(21) Appl. No.: 16/684,297

(22) Filed: Nov. 14, 2019

Related U.S. Application Data
(60) Provisional application No. 62/767,366, filed on Nov. 14, 2018.

(51) Int. Cl. A61F 2/44

(52) U.S. Cl. CPC A61F 2/4455; A61F 2002/30985; A61F 2310/00023

(57) ABSTRACT

Disclosed herein are devices, systems, and methods for securing a strut graft to a plate for enhancing the security and stability of a spinal fusion after corpectomy. The disclosed strut grafts include at least one anchoring device positioned within the interior of the strut graft that are configured to securedly engage a screw that passes through the plate before engaging the anchoring device. The disclosed methods, systems, and devices may aid in preventing the strut graft from migrating in the intervertebral space after surgery.

### CROSS REFERENCE TO RELATED APPLICATIONS

This application claims the benefit of priority pursuant to 35 U.S.C. § 119(e) of U.S. provisional patent application No. 62/767,366, filed 14 Nov. 2018, entitled “Devices, Systems, And Methods For Stabilization Of Intervertebral Cages,” which is hereby incorporated by reference herein in its entirety.

### FIELD

The disclosed processes, methods, and systems are directed to methods, systems, and devices for fixedly attaching intervertebral cages to locking plates.

### BACKGROUND

Over the past 15 years spinal fusion has become a ubiquitous treatment modality for degenerative spinal condition, deformity, infection, and tumor resections. One of the most recent advancements is the use of 3D printed titanium cages. Rapidly, these cages are replacing older modalities such as allograft bone and Polyetheretherketone (PEEK) cages.

### SUMMARY

Disclosed herein are various devices for securing a plate to an intervertebral cage. The devices comprising, an anchoring device may define an opening for securedly receiving a screw, bolt, clamp, cord or other fastener, and an intervertebral cage having an exterior surface and an interior, wherein the cage may include at least one window for at least allowing a screw, bolt, clamp, cord or other fastener to pass through the exterior surface into the interior, wherein the interior of the cage may be configured to hold the anchoring device, and a plate having at least one window configured to align with the window of the cage, and at least two windows for allowing at least two screws, bolts, clamps, cords or other fastener to pass through the plate. In some embodiments, the anchoring device may be integrated into the cage during fabrication of the cage, and the window in the cage may be configured to allow the anchoring device to pass from through the exterior surface of the cage and be positioned within the interior of the cage.

Also disclosed herein are systems for fixedly attaching a plate to an intervertebral cage. The systems disclosed herein may comprise a plate, an anchoring device, an intervertebral cage, and at least one screw, bolt, clamp, cord or other fastener configured to pass through the plate and engage the anchoring device.

Also disclosed are methods of securing an intervertebral strut graft in a corpectomy, the methods comprising the steps of placing a strut graft cage in an intervertebral space, securing an anchoring device positioned within the interior of the cage with a screw, bolt, clamp, cord or other fastener wherein the screw passes through a plate, and securing the plate to an intervertebral body on each side of the intervertebral space.

Also disclosed herein are methods of maintaining the position of an intervertebral strut graft, the methods comprising steps of placing a strut graft cage in an intervertebral space, securing an anchoring device positioned within the interior of the cage with at least one fastener, wherein the fastener passes through a plate, securing the plate to a first intervertebral body above the space and a second intervertebral body below the space; and thereby maintaining the position of the strut graft. In some embodiments, the methods may include a device wherein at least one fastener is selected from a screw, bolt, clamp, cord or other fastener. In various embodiments, the methods may be useful where the first intervertebral body is a cervical vertebra, or a thoracic vertebra, or a lumbar vertebra, and the strut graft cage may, in some embodiments be a printed cage, for example a cage comprising titanium. The cage of the device may, in some embodiments, comprise an integral anchoring device for receiving at least a first fastener, and the anchoring device may be inserted into the cage prior to insertion of the anchoring device into a patient. In many embodiments, the disclosed device helps to prevent the strut graft from migrating from its position after being secured to the plate.

### BRIEF DESCRIPTION OF THE DRAWINGS

FIG. 1 depicts a frontal (AlP) the disclosed construct. A strut graft (solid parallel lines) is shown positioned between two vertebral bodies (squares). The dashed square depicts a single vertebral body that has been removed. The plate is shown in blue, with two screws each affixing the plate to (1 and 2) the adjacent vertebral bodies and (3) the strut graft.

FIG. 2 depicts a side (sagittal), cross-sectional view of the disclosed construct of FIG. 1. The strut graft is depicted as a blue square fixedly attached to the plate (also blue) by a screw. Screws are also shown anchoring the plate to the adjacent vertebral bodies.

FIG. 3 depicts embodiments of the disclosed devices and systems for one (upper panel), two (middle panel), and three (lower panel) corpectomy. These embodiments show two slots and anchoring devices per level, but some embodiments may comprise fewer (one), or more than two per level.

FIG. 4 is an axial cross-sectional view of one embodiment of the disclosed devices and systems. In this embodiment, the strut graft is shown with a circular cross-section, and two anchoring devices are engaged with two screws that secure the plate to the strut graft.

FIG. 5 is a detailed view showing two windows for the anchoring device that allow for both caudal and cephalad movement, as well as side-to-side movement. Also depicted is one embodiment of an anchoring device that allows the surgeon to precisely angle the screws during positioning.

FIG. 6 depicts an embodiment of the disclosed device and system with a single screw engaging a single anchoring device integrated into the body of the strut graft.

FIG. 7 depicts an embodiment of the disclosed device and system wherein the strut graft includes a window for insertion of the anchoring device after fabrication. This allows the anchoring device to be inserted, aligned, and adjusted, if necessary, by the surgeon. This embodiment may be used with single screw or multi screw embodiments.

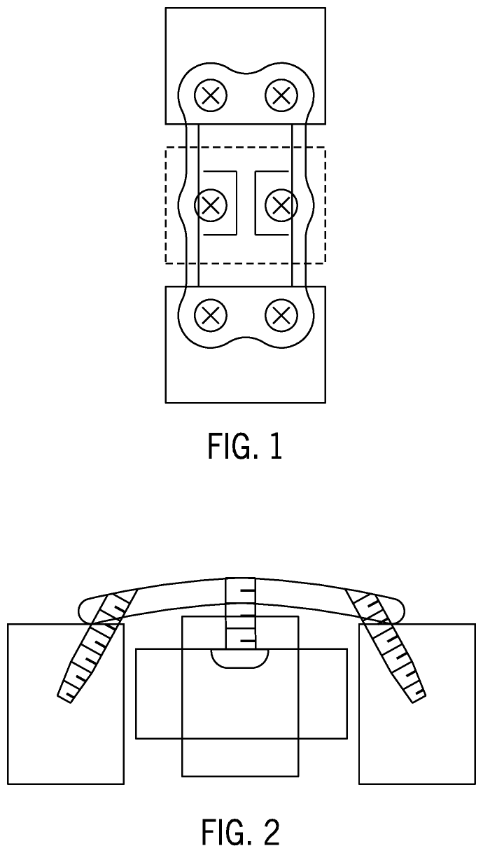

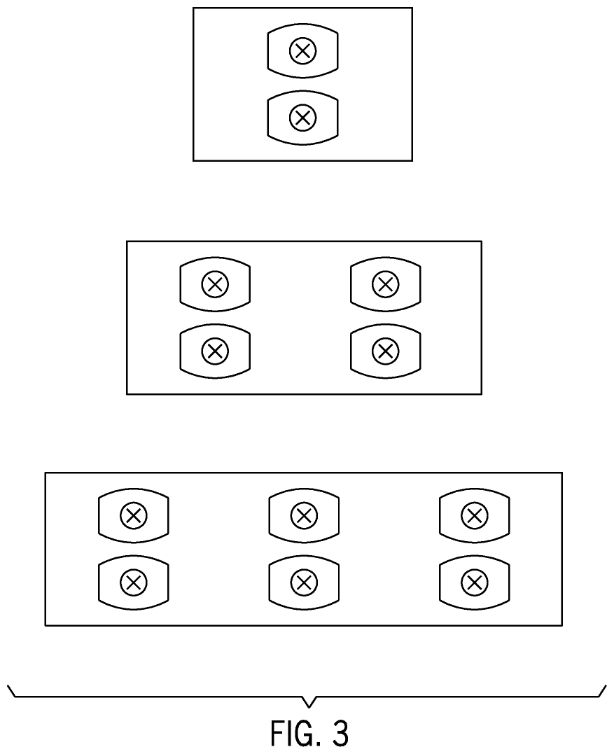

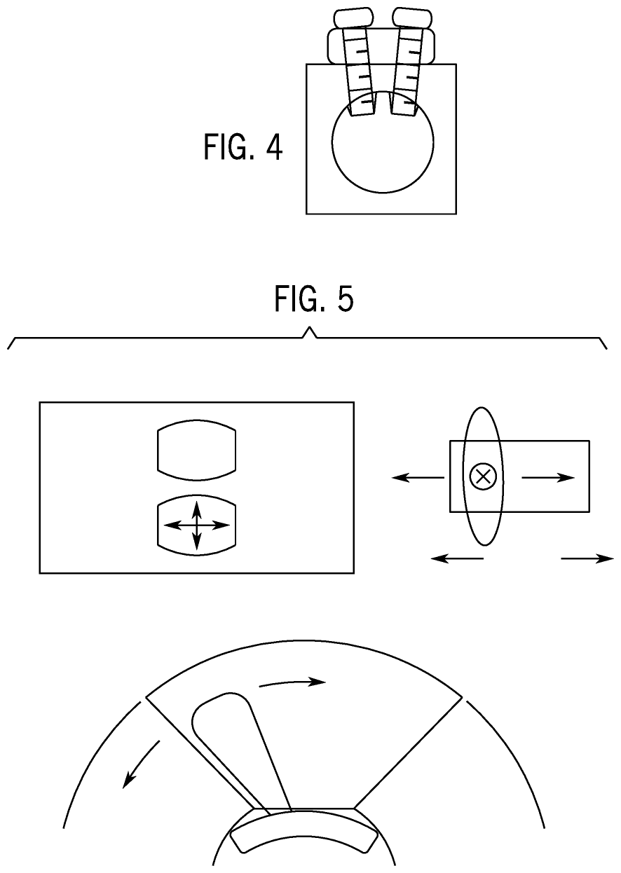

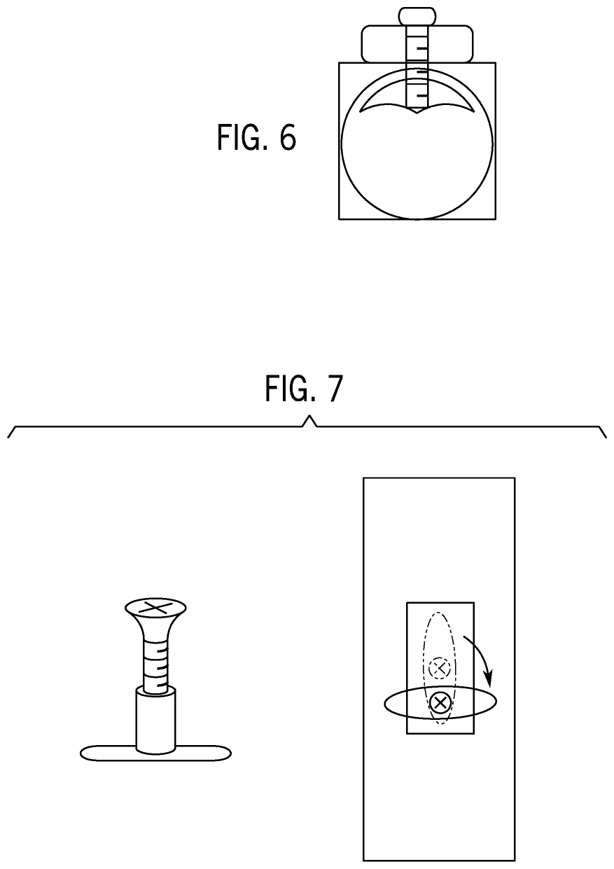

### DETAILED DESCRIPTION

Disclosed herein are devices and systems for fixedly connecting segmental cages and plates in various spinal fusion procedures. The disclosed devices, systems, and methods may be used in fusions in the cervical, thoracic, or lumbar region of the spine, and may aid in preventing movement or migration of the cages after surgery. In some embodiments, the disclosed device is an anchoring device that helps anchor segmental cages to the plate. The disclosed anchoring device may be integrated into the segmental cage or implanted into the cage structure after the cage is in place, or just prior to placing the cage into the intervertebral space.

Traditionally, corpectomy cages are used to replace at least two intervertebral discs and one vertebral body after these structures have been removed. The space formed by removal of these structures has typically been filled with a strut graft implant that sits against the endplates of the adjacent vertebral bodies. A titanium plate is placed on the surface of the adjacent vertebral bodies and spans the space occupied by the strut graft implant. The plate provides additional strength and support. These two implants, the strut graft and titanium plate, are usually distinct, separate, and unconnected.

Disclosed herein are devices, systems, and methods that allow the strut graft to be fixedly connected to the titanium plate. In most embodiments, the disclosed devices and systems include an anchoring device that is integrated into the segmental cage during its fabrication. Alternatively, the anchoring device may be added to the segmental cage after its fabrication. By utilizing the anchoring device to fixedly connect the plate and strut graft to create a construct, the construct can act as one structure giving enhanced stability and strength to the entire construct. This connection improves the safety of the construct by anchoring the vertebral strut graft to the titanium plate, and minimizing the chance of strut migration. Minimizing or preventing migration may result in reduction or prevention of neural, vascular, or other trauma that can cause serious injury to a patient. Traditional therapies, for example in cervical corpectomies, may result in migration in 1-3% of procedures. These rates may increase where multilevel procedures are performed. Additionally, the disclosed devices, systems, and methods may provide benefits where there would be load sharing by the entire construct that would help alleviate subsidence or telescoping of the strut graft into the adjacent vertebral bodies. This would also aid in the bone biology and physiology enhancing the overall fusion construct.

An anchoring device can be designed into the strut graft during fabrication. In these embodiments, the surgeon may use existing screw holes, for example screw holes in the strut graft that normally would be used to place screws into an adjacent vertebral body to anchor to the graft. In these embodiments, a screw or other fastener is inserted through the plate into the strut graft to engage the integrated anchoring device. Connector or fastener devices other than screws may also be used with the disclosed devices, systems, and methods, for example the connector may be a rigid or flexible fastener, a mechanical fastener, chemical fastener, interference-fit, etc. for example pin, bolt, tie, clamp, clip, chain, rope, cord, line, tether, rivet, glue, adhesive, polymers, etc.

In some embodiments, the anchoring device, strut graft, or plate may allow some variability of alignment. In these embodiments, the alignment variability may provide for some movement by the graft and/or anchoring device so that a surgeon could align the screw holes with the anchoring device. In most embodiments, positions may be varied by between about 0.1 and 10 mm, for example greater than about 0.1 mm, 0.2 mm, 0.3 mm, 0.4 mm, 0.5 mm, 0.6 mm, 0.7 mm, 0.8 mm, 0.9 mm, 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, or 10.0 mm, and less than about 10.0 mm, 9.5 mm, 9.0 mm, 8.5 mm, 8.0 mm, 7.5 mm, 7.0 mm, 6.5 mm, 6.0 mm, 5.5 mm, 5.0 mm, 4.5 mm, 4.0 mm, 3.5 mm, 3.0 mm, 2.5 mm, 2.0 mm, 1.5 mm, 1.0 mm, 0.9 mm, 0.8 mm, 0.7 mm, 0.6 mm, 0.5 mm, 0.4 mm, 0.3 mm, or 0.2 mm. Positions may varied in x-, y-, and/or z-axis such as the frontal, longitudinal, or sagittal axes.

In some embodiments, a specifically machined fastener could be used or alternatively the anchoring device may accept standard fastener (for example screws, bolt, clamp, cord, etc.) designed to be placed into the vertebral body.

The disclosed anchoring device may include a fastener of various types, materials, and sizes. The disclosed fastener may be rigid or flexible. In some embodiments, the fastener may be a screw, bolt, clamp, or similar structure that may aid in securedly fixing and/or altering the position of the cage or strut relative a plate. Flexible fasteners for use with the disclosed devices may also be used, for example a cord or flexible rope, line, tether, etc. that may be held in place, for example under tension, between the cage and the plate. In many embodiments, the disclosed device may include more than one anchoring device, wherein the anchoring devices may be the same or different—such as a first anchoring device comprising a screw, and a second anchoring device comprising a cord. As noted above, the anchoring device may be embedded in the cage at the time of manufacture of the device or it can be added at the time of surgery. Likewise, the fastener may be incorporated into the device during or after manufacture, for example during surgery. Alternatively, some embodiments may include a toggle device or expanding anchor system that may be incorporated with the anchoring device and/or be separate from the plate or cage. In some embodiments, the disclosed devices may be incorporated into existing plate and screw combinations, for example those having a suitable anchoring point within the cage. In many embodiments, screw holes may be positioned within the plate and can be used as the anchoring point at or through the plate. In various embodiments, other anchoring points may be used as well. This includes any design features within the plate that are specifically designed to accommodate the system that anchors a cage to the plate.

An anchoring device could also be placed into the cage after the strut and titanium plates are in place. In some embodiments, the anchoring device may include one or more structures that may aid in contacting the strut or cage, for example fin structures that can be rotated once the anchoring device has been positioned inside the strut, for example a 3D titanium strut. In these embodiments, rotating the screw may pull the anchoring device toward the plate—thus securing or fixing the position of the two apparatus relative to each other.

Either of these methods establishes a secure anchoring point to the titanium strut graft establishing all of the benefits afforded by securing the plate and the titanium strut graft together.

A spacer may be positioned between the strut graft and plate to maintain the position of the cage when the fastener is attached and/or tightened. In some embodiments, the cage may be fabricated with an integral spacer, while other embodiments may allow for the surgeon to select an appropriate spacer to maintain the proper strut graft alignment.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. A device for securing a plate to a intervertebral cage comprising:
an anchoring device defining an opening for securedly receiving a screw, bolt, clamp, cord or other fastener;
an intervertebral cage having an exterior surface and an interior, wherein the cage includes at least one window for at least allowing a screw, bolt, clamp, cord or other fastener to pass through the exterior surface into the interior, wherein the interior of the cage is configured to hold the anchoring device; and
a plate having at least one window configured to align with the window of the cage, and at least two windows for allowing at least two screws, bolts, clamps, cords or other fastener to pass through the plate.

2. The device of claim 1, wherein the anchoring device is integrated into the cage during fabrication of the cage.

3. The device of claim 2, wherein the window in the cage is configured to allow the anchoring device to pass from through the exterior surface of the cage and be positioned within the interior of the cage.

4. A system for fixedly attaching a plate to an intervertebral cage comprising:
a plate;
an anchoring device;
an intervertebral cage; and
at least one screw, bolt, clamp, cord or other fastener configured to pass through the plate and engage the anchoring device.

5. A method of securing an intervertebral strut graft in a corpectomy comprising the steps of;
placing a strut graft cage in an intervertebral space;
securing an anchoring device positioned within the interior of the cage with a screw, bolt, clamp, cord or other fastener wherein the screw passes through a plate;
securing the plate to an intervertebral body on each side of the intervertebral space.

6. A method of maintaining the position of an intervertebral strut graft, the method comprising the steps of;
placing a strut graft cage in an intervertebral space;
securing an anchoring device positioned within the interior of the cage with at least one fastener, wherein the fastener passes through a plate;
securing the plate to a first intervertebral body above the space and a second intervertebral body below the space; and thereby
maintaining the position of the strut graft.

7. The method of claim 6, wherein the at least one fastener is selected from a screw, bolt, clamp, cord or other fastener.

8. The method of claim 6, wherein the first intervertebral body is a cervical vertebra.

9. The method of claim 6, wherein the first intervertebral body is a thoracic vertebra.

10. The method of claim 6, wherein the first intervertebral body is a lumbar vertebra.

11. The method of claim 6, wherein the strut graft cage is a printed cage.

12. The method of claim 9, wherein the cage comprises titanium.

13. The method of claim 10, wherein the cage comprises an integral anchoring device for receiving at least a first fastener.

14. The method of claim 9, wherein an anchoring device is inserted into the cage prior to insertion of the anchoring device.

15. The method of claim 6, wherein the strut graft does not migrate from its position after being secured to the plate.
